(19) United States
(12) Patent Application Publication
(10) Pub. No.: US 2008/0167976 A1
(43) Pub. Date: Jul. 10, 2008

(54) MANAGEMENT SYSTEM AND METHOD FOR A BIOSTRIP

(75) Inventors: Ya-Hsin Chuang, Taipei (TW); Meng-Yi Lin, Taipei (TW)

Correspondence Address: WPAT, PC;INTELLECTUAL PROPERTY ATTORNEYS, 2030 MAIN STREET, SUITE 1300, IRVINE, CA, 92614, US

(73) Assignee: HEALTH & LIFE CO., LTD., Taipei (TW)

(21) Appl. No.: 11/782,536

(22) Filed: Jul. 24, 2007

(30) Foreign Application Priority Data
Jan. 5, 2007 (TW) 096100481

(51) Int. Cl. G06Q 10/00

(52) U.S. Cl. 705/28

(57) ABSTRACT

The present invention discloses a management system and method for a biostrip. The management method is applicable to a biomedical measurement device to manage at least one biostrip. The management method comprises the following steps: inputting at least one piece of setting data of the biostrip; generating a count reference value based on the time of the inputted setting data, data content or combinations thereof; monitoring the expiration date of the biostrip corresponding to the count of the count reference value, and it further comprises displaying the current date, the expiration date of the biostrip, the amount of strips, the first opening date or combinations thereof. When the expiration date of the biostrip has passed, a user is then reminded to avoid using the expired biostrip.

### BACKGROUND OF THE INVENTION

1. Field of the Invention

The present invention provides a management system and method for a biostrip. In particular, the invention is directed to the management of the expiration dates of biostrip so as to avoid using an expired biostrip for testing.

2. Description of the Prior Art

Many test devices for the determination of certain analytes in biological fluids have been developed. Biostrips for more convenient testing have also been developed so that tests can be done in hospitals, clinical laboratories, clinics or at home at any time in order to observe a subject's health condition. Biosensors are used to determine various physiological indexes of an organism such as glucose, cholesterol or uric acid and the like. Disposable test strips are often used and special measuring techniques are applied to obtain the results for the subjects to be tested. The quality of the biostrip is one of the essential factors that influences the preciseness of the test. Generally, when the test strips are made, the manufacturer will give calibration data to the same batch of test strips based on the chemical and physical properties thereof to obtain a more precise analytical result. However, the chemical and physical properties of the test strips are subject to changes with time due to influences from environmental factors, so errors or invalid analytical results might be obtained. Therefore, in addition to cautiousness regarding the sealing and preservation of biostrips, “shelf life” and “opening shelf life” are also given thereto, both of which are managed by the user himself. The counting of “shelf life” starts upon the manufacture of test strips and the starting date for “opening shelf life” depends on when the user first opens the sealed package for use. In many cases, expired test strips are used unknowingly due to the user's negligence, resulting in errors in the analytical results and therefore influencing the determination of the health condition of the subject. For convenience, “expiration date” is used herein to represent the earlier date of the expiration date of either between “shelf life” and “opening shelf life.”

To meet the need of improving the management of the expiration date, the inventors of the present invention have proposed a management system and method for a biostrip based on years of research and practical experiences to practice accordingly.

### SUMMARY OF THE INVENTION

In view of the above, the object of the invention is to provide a management system and method for a biostrip. In particular, the invention is directed to the management of the expiration date of a biostrip so as to avoid using the expired biostrip for testing.

Therefore, to achieve the above object, the management method for the biostrip of the invention applicable to a biomedical measurement device to manage at least one biostrip comprises the steps of:
(1) inputting at least one piece of data of the biostrip;
(2) generating a count reference value based on the time of the inputted setting data, data content or combinations thereof; and
(3) monitoring the expiration date of the biostrip corresponding to the count of the count reference value.

The management method may further comprise the following steps:
(4) displaying the current date, the expiration date of the biostrip, the amount of biostrips in a batch, the first opening date or combinations thereof;
(5) reminding the user when the monitored biostrip has passed the count reference value.

According to the management method for the biostrip of the invention, a management system is further provided, which comprises at least a setting unit, a management unit and an execution unit. The setting unit is used for inputting at least one piece of setting data of the biostrip. The management unit is for generating a count reference value based on the time of the inputted setting data, data content or combinations thereof and for counting. The execution unit is used for monitoring the expiration date of the biostrip corresponding to the count of the count reference value. The management of the expiration dates is therefore performed to avoid using the expired biostrip for testing.

The technical characteristics of the invention and the effects achieved thereby are further illustrated by the following detailed description in conjunction with the preferred embodiments and the relevant drawings.

### BRIEF DESCRIPTION OF THE DRAWINGS

FIG. 1 is a block diagram showing a management system for the biostrip of the invention;

FIG. 2 is a block diagram showing another management system for the biostrip of the invention;

FIG. 3 is a schematic diagram showing the management method for the biostrip of the invention; and

FIG. 4 is a working scheme showing the management method for the biostrip of the invention.

### DETAILED DESCRIPTION OF THE INVENTION

To further explicate the above objects, characteristics and advantages of the invention, the biostrip of the invention will be described in detail in the following section with reference to a preferred embodiment in conjunction with the relevant drawings, wherein one element will be described with one element numeral.

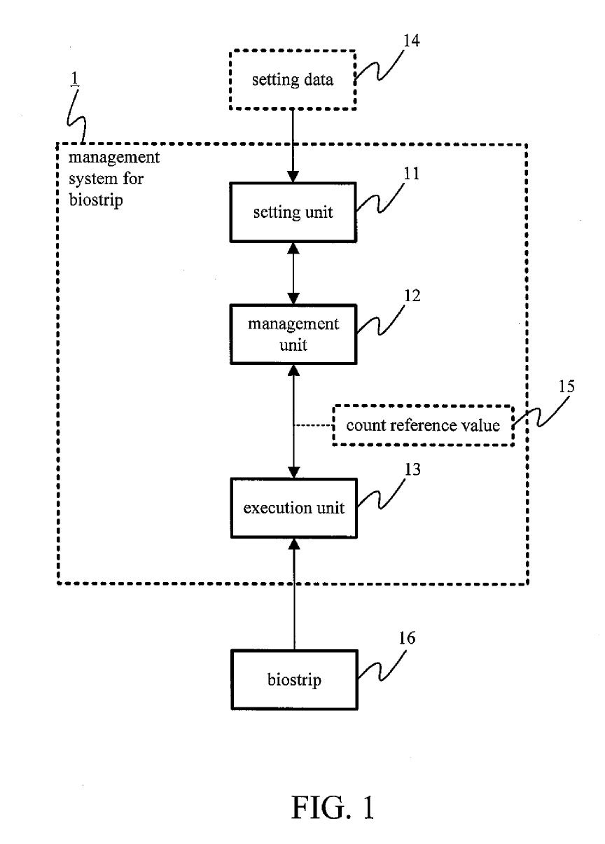

FIG. 1 is a block diagram showing a management system for the biostrip of the invention, wherein the management system 1 is applicable to a biomedical measurement device to manage at least one biostrip 16. The management system 1 comprises at least a setting unit 11, a management unit 12 and an execution unit 13. The setting unit 11 is used for inputting at least one piece of setting data 14 of the biostrip 16. The management unit 12 is for generating a count reference value 15 based on the time of the inputted setting data, data content or combinations thereof and for counting. The execution unit 13 is used for monitoring the expiration date of the biostrip 16 corresponding to the count of the count reference value 15.

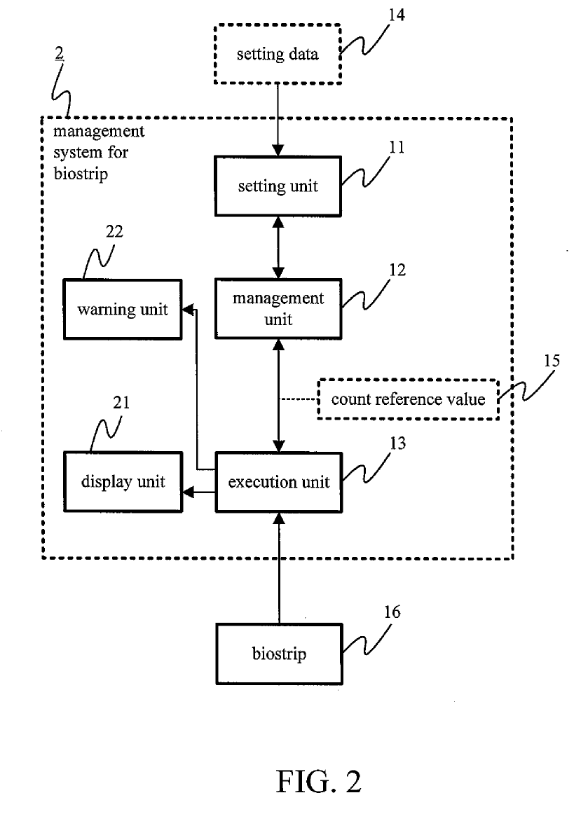

FIG. 2 is a block diagram showing another management system for the biostrip of the invention, wherein the management system 2 is applicable to a biomedical measurement device to manage at least one biostrip 16. The management system 2 comprises at least a setting unit 11, a management unit 12, an execution unit 13, a display unit 21 and a warning unit 22. The setting unit 11 is used for inputting at least one piece of setting data 14 of the biostrip 16. The management unit 12 is for generating a count reference value 15 based on the time of the inputted setting data, data content or combinations thereof and for counting. The execution unit 13 is used for monitoring the expiration date of the biostrip 16 corresponding to the count of the count reference value 15. The display unit 21 is for displaying the current date, the expiration date of the biostrip 16 monitored by the execution unit 13, the amount of biostrip in a batch, the first opening date or combinations thereof. The warning unit 22 is for reminding the user when the biostrip 16 monitored by the execution unit 13 has passed the count reference value.

The setting data 14 preferably includes the calibration code, the calibration data, the operation parameters, the amount of the biostrip in a batch, the first opening date, the expiration date or combinations thereof, wherein the setting data can be provided by inserting a memory card or by obtaining the setting data in the biomedical measurement device after manually setting. The count reference value 15 preferably includes the expiration date, the number of days or the number of weeks to the expiration date, the amount of strips or combinations thereof. The time of inputting and/or changing the setting data 14 is used as the first opening date of the test strip and to start counting the “opening shelf life” thereof or to start counting the amount of strips used. When the accumulated amount of the used strips is over the amount of strip in the count reference value 15, the user is reminded to reset the setting data 14. The management unit 13 further comprises a counting unit for counting time or for counting amount.

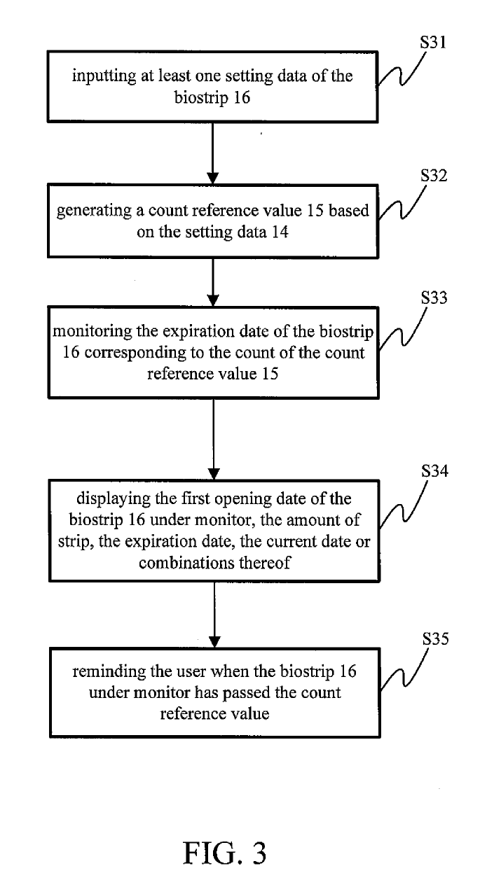

FIG. 3 is a schematic diagram showing the management method for the biostrip of the invention. The management method is applicable to a biomedical measurement device to manage at least one biostrip. The steps of the management method are as follows:

Step S31: inputting the setting data 14 of the biostrip 16;

Step S32: generating a count reference value 15 based on the time of the inputting or changing setting data 14, data content or combinations thereof;

Step S33: monitoring the expiration date of the biostrip 16 corresponding to the count of the count reference value 15;

Step S34: displaying the current date, the expiration date of the monitored biostrip 16, the amount of strips, the first opening date or combinations thereof; and

Step S35: reminding the user when the monitored biostrip 16 has passed the count reference value.

The setting data 14 preferably includes the calibration code, the calibration data, the operation parameters, the amount of the biostrip 16 in a batch, the first opening date, the expiration date, or combinations thereof, wherein the setting data can be provided by inserting a memory card or by obtaining the setting data in the biomedical measurement device after manually setting. The count reference value 15 preferably includes the expiration date, the number of days or the number of weeks to the expiration date, the amount of strips or combinations thereof. The time of inputting and/or changing the setting data 14 is used as the first opening date of the test strip and to start counting the “opening shelf life” thereof or to start counting the used amount of strips. When the accumulated amount of the used strips is over the amount of strips in the count reference value 15 in the setting data 14, the user is reminded to reset the setting data 14. The management unit 13 further comprises a counting unit for counting time or for counting amount.

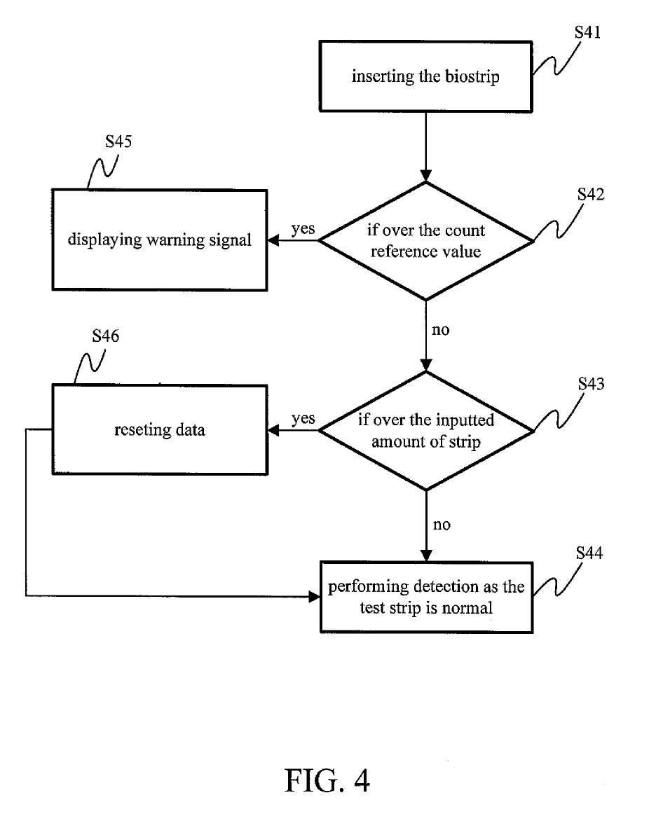

FIG. 4 is a working scheme showing the management method for the biostrip of the invention. When the biostrip 16 is inserted in the biomedical measurement device, the working scheme of the management method for the biostrip of the invention is performed. The count reference value 15 used in the subject example preferably includes the expiration date, the number of days, the number of weeks or combinations thereof. The steps of the scheme are as follows:

Step S41: inserting the biostrip;

Step S42: determining if the current date has passed the expiration date, the number of days, the number of weeks or combinations thereof and then executing Step 45 if the answer is yes and executing Step S43 if the answer is no;

Step S43: determining if the number of strips from the accumulated counting is over the inputted amount of strips and then executing Step 46 if the answer is yes and executing Step S44 if the answer is no;

Step S44: performing detection to determine whether the quality of the test strip is normal;

Step S45: displaying a warning signal; and

Step S46: displaying a reminding signal to reset the setting data and then executing Step S44.

Generally, the warning signal can be a signal of image, sound, light, vibration and the like to warn the user that the biostrip is expired.

The above description is provided for illustration only. It can be in no way construed as a limitation to the present invention. Any modifications or changes made by any skilled person in the art according to the disclosure of the invention without departing from the spirit of the invention are intended to be within the scope of the invention as defined by the appended claims.

### BRIEF DESCRIPTION OF THE ELEMENT NUMERALS

1: management system for biostrip;
2: management system for biostrip;
11: setting unit;
12: management unit;
13: execution unit;
14: setting data;
15: count reference value;
16: biostrip;
21: display unit;
22: warning unit;
S31-S35: scheme steps; and
S41-S46: scheme steps.

What is claimed is:

1. A management system for a biostrip applicable to a biomedical measurement device to manage at least one biostrip, said management system at least comprising:
a setting unit, which is used for inputting at least one piece of setting data of the biostrip.
a management unit, which is for generating a count reference value based on the time of the inputted setting data, data content or combinations thereof and for counting; and
an execution unit, which is used for monitoring the expiration date of the biostrip corresponding to the count of the count reference value.

2. The management system for the biostrip of claim 1, wherein the setting data preferably includes the calibration code, the calibration data, the operation parameters, the amount of the biostrip in a batch, the first opening date, the expiration date or combinations thereof.

3. The management system for the biostrip of claim 1, wherein the count reference value includes the expiration date, the number of days, the number of weeks to the expiration date or combinations thereof.

4. The management system for the biostrip of claim 1, wherein the management unit comprises a counting unit for the counting of time, amount or combinations thereof.

5. The management system for the biostrip of claim 1, wherein the execution unit determines the status of the strip by comparing the count reference value with the data of the current time.

6. The management system for the biostrip of claim 2, wherein the accumulated amount of the used biostrips is counted according to the inputted amount of biostrips and a reminder for resetting data will be provided when the accumulated amount is over the inputted amount of strips.

7. The management system for the biostrip of claim 1, wherein the management system further comprises a warning unit, which is for reminding the user when the biostrip monitored by the execution unit has passed the count reference value.

8. The management system for the biostrip of claim 3, wherein the management system further comprises a display unit, which is for displaying the current date, the expiration date of the biostrip monitored by the execution unit, the amount of strips, the first opening date or combinations thereof.

9. A management method for a biostrip applicable to a biomedical measurement device to manage at least one biostrip, said management method at least comprising:
inputting at least one piece of setting data of the biostrip;
generating a count reference value based on the time of the inputted setting data, data content or combinations thereof; and
monitoring the expiration date of the biostrip corresponding to the count of the count reference value.

10. The management method for the biostrip of claim 9, wherein the count reference value 15 preferably includes the expiration date, the number of days, the number of weeks or combinations thereof.

11. The management method for the biostrip of claim 9, wherein the setting data preferably includes the calibration code, the calibration data, the operation parameters, the amount of the biostrip 16 in a batch, the first opening date or the expiration date, or combinations thereof.

12. The management method for the biostrip of claim 9, wherein the count reference value includes the expiration date, the number of days, the number of weeks to the expiration date or combinations thereof.

13. The management method for the biostrip of claim 9, wherein the monitoring of the expiration date of the biostrips includes the counting of time, amount or combinations thereof.

14. The management method for the biostrip of claim 9, wherein the status of the strip is determined by comparing the count reference value with the data of the current time.

15. The management method for the biostrip of claim 9, wherein the accumulated amount of the used biostrips is counted according to the inputted amount of biostrips and a reminder to reset the first opening date, the amount of strips or the combination thereof will be provided when the accumulated amount is over the inputted amount of strips.

16. The management method for the biostrip of claim 9, wherein the management method further comprises reminding the user when the monitored biostrip has passed the count reference value.

17. The management method for the biostrip of claim 9, wherein the management method further comprises displaying the current date, the expiration date of the monitored biostrip, the amount of strips, the first opening date or combinations thereof.
